(19) United States
(12) Patent Application Publication
(10) Pub. No.: US 2016/0354017 A1
(43) Pub. Date: Dec. 8, 2016

(54) SYSTEMS AND METHODS FOR SENSOR CALIBRATION IN PHOTOPLETHYSMOGRAPHY

(71) Applicant: Covidien LP, Mansfield, MA (US)

(72) Inventors: Christopher J. Meehan, Golden, CO (US); Daniel Lisogurski, Boulder, CO (US); Charles Keith Haisley, Boulder, CO (US)

(21) Appl. No.: 15/242,033

(22) Filed: Aug. 19, 2016

Related U.S. Application Data
(63) Continuation of application No. 13/842,171, filed on Mar. 15, 2013.

(51) Int. Cl. A61B 5/1495; A61B 5/00; A61B 5/1455

(52) U.S. Cl. CPC A61B 5/1495; A61B 5/14552; A61B 5/0002; A61B 2560/0223; A61B 2562/0238

(57) ABSTRACT

Various methods and systems for obtaining calibration coefficients for pulse oximeter sensors are provided. A method includes passing current through a light emitting element in an oximeter sensor and measuring, utilizing a first voltage sensing lead, a first voltage present at an electrical input of the light emitting element. The method also includes measuring, utilizing a second voltage sensing lead, a second voltage present at an electrical output of the light emitting element and determining a forward voltage of the light emitting element based on the first and second voltages. Utilizing the determined forward voltage, a wavelength of light emitted from the light emitting element is calculated. Utilizing the calculated wavelength of the emitted light, at least one calibration coefficient for the oximeter sensor is determined.

### CROSS-REFERENCE TO RELATED APPLICATION

This application is a continuation of U.S. patent application Ser. No. 13/842,171, entitled “Systems and Methods for Sensor Calibration in Photoplethysmography,” filed Mar. 15, 2013, the disclosure of which is hereby incorporated by reference in its entirety for all purposes.

### BACKGROUND

The present disclosure relates generally to pulse oximetry and, more particularly, to oximeter sensor calibration systems and methods.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, medical practitioners often desire to monitor certain physiological characteristics of their patients. Accordingly, a wide variety of devices have been developed for monitoring physiological characteristics. Such devices provide doctors and other healthcare personnel with the information they need to provide healthcare for their patients. As a result, such monitoring devices have become an indispensable part of modern medicine. One technique for monitoring certain physiological characteristics or parameters of a patient is commonly referred to as photoplethysmography (PPG). PPG is an optical technique that can be used to non-invasively detect blood volume changes in the microvascular bed of a patient's tissue by taking measurements at the skin surface, and these measurements may be utilized to calculate physiological parameters such as heart rate, cardiac arrhythmia, respiration rate, respiration effort, fluid responsiveness, blood pressure, and so forth. One type of PPG system is commonly referred to as pulse oximetry, and the devices built based upon pulse oximetry techniques are commonly referred to as pulse oximeters.

A pulse oximeter is typically used to measure various physiological characteristics, such as the blood oxygen saturation of hemoglobin in arterial blood of a patient. Blood oxygen saturation is typically estimated as a ratio of oxygenated hemoglobin to deoxygenated hemoglobin present in the patient's tissue. Hemoglobin is the component of blood which transports oxygen throughout the body. The ratio of oxygenated hemoglobin to deoxygenated hemoglobin can be determined by directing light at certain wavelengths into the patient's tissue and measuring the absorbance of the light. In certain systems, a first wavelength of light may be selected at a point in the electromagnetic spectrum where the absorption of oxygenated hemoglobin differs from the absorption of deoxygenated hemoglobin. A second wavelength may be selected at a different point in the spectrum where the light absorption differs from absorption at the first wavelength. Thus, such light can be passed through a patient's tissue, and the amount of absorption of the light at each wavelength can be used to determine the relative amounts of oxygenated and deoxygenated hemoglobin in the patient's blood. For example, wavelength selections for measuring normal blood oxygenation levels typically include a red light emitted at approximately 660 nanometers (nm) and a near-infrared light emitted at approximately 900 nm.

One method for estimating blood oxygen saturation is to calculate a characteristic known as the ratio-of-ratios (Ratrat) of the absorption of red light (RED) to near-infrared light (IR). While various methods may be utilized to calculate Ratrat, in one method, a sensor is used to emit red and near-infrared light into a patient's tissue and detect the light that is reflected back. Signals indicative of the detected light are conditioned and processed to generate plethysmographic waveforms. The plethysmographic waveforms typically have a pulsatile component as well as components that change slower than the heart rate of the patient. Taken together, these components of the RED wavelength and IR wavelength signals may then be used to calculate Ratrat, which has been observed to correlate well to blood oxygen saturation. This observed correlation may be used to estimate blood oxygen saturation based on the measured value of the Ratrat.

Therefore, pulse oximeters may measure Ratrat in order to determine blood oxygen saturation. The relationship between Ratrat and blood oxygen saturation may follow a line that serves as a sensor calibration curve. Because the light absorption of the blood's oxygenated hemoglobin and deoxygenated hemoglobin is wavelength-dependent, the particular calibration curve that correlates Ratrat to blood oxygen saturation depends upon the specific wavelength of the light emissions by the sensor's light emitting diodes (LEDs). Thus, the particular wavelength emitted by the LED affects not only the measured Ratrat, but also the calibration curve that correlates that Ratrat to blood oxygen saturation. Shifting the wavelength of the emitter may cause the sensor's calibration curve to be shifted and rotated. Therefore, measurements of blood oxygen saturation (and other desired physiological parameters) may be more accurate when the sensor's calibration curve corresponds to the actual wavelengths of the sensor's LEDs.

For this reason, pulse oximeter sensors may include a digital memory chip that stores calibration information related to the wavelength of the LEDs in the sensor. Unfortunately, this digital memory chip is often of high monetary cost. Additionally, in some instances, the wavelength of the LED may shift during operation based on implementation-specific factors. For example, in certain systems, the wavelength of the LED may change based on temperature changes in the system, and the calibration curve stored on the digital memory chip may no longer be accurate for the current operation of the LED. Accordingly, there exists a need for calibration systems and methods that identify calibration curves for sensors.

### BRIEF DESCRIPTION OF THE DRAWINGS

Advantages of the disclosed techniques may become apparent upon reading the following detailed description and upon reference to the drawings in which:

FIG. 1A illustrates an embodiment of a wireless patient monitoring system including a patient monitor and a sensor;

FIG. 1B illustrates an embodiment of a wireless patient monitoring system including a patient monitor, a dongle, and a sensor;

FIG. 2A illustrates an embodiment of a wired patient monitoring system including a patient monitor and a sensor;

FIG. 2B illustrates an embodiment of a wired patient monitoring system including a patient monitor, a dongle, and a sensor;

FIG. 3 is a block diagram illustrating an embodiment of a wireless patient monitoring system including a sensor and a pulse oximeter;

FIG. 4 is a block diagram illustrating an embodiment of a wired patient monitoring system including a sensor and a pulse oximeter;

FIG. 5A illustrates an embodiment of a circuit capable of determining a forward voltage of a light source included in a sensor of a patient monitoring system;

FIG. 5B illustrates an alternate embodiment of the circuit of FIG. 5A having a switch for coupling and decoupling of wavelength measurement components to the light source;

FIG. 6 is a flow chart illustrating an embodiment of a method of operating the circuit of FIG. 5 to obtain calibration information for a sensor of a patient monitoring system;

FIG. 7 is a flow chart illustrating an embodiment of a method of operation of a controller that may be implemented to determine a calibration curve for a sensor;

FIG. 8 is a flow chart illustrating an embodiment of a method for initiating and calibrating a patient monitoring system; and

FIG. 9 is a schematic illustrating an embodiment of a calibration retrofit kit for retrofitting an oximetry sensor.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described in detail below, provided herein are embodiments of calibration methods and systems for photoplethysmography systems, such as pulse oximetry systems. In the discussion below, the calibration systems and methods are discussed in the context of a pulse oximetry system to facilitate explanation, but presently disclosed embodiments are contemplated to have utility in a wide range of types of photoplethysmography systems and are not limited to pulse oximetry applications. With this understanding, presently disclosed embodiments include one or more features that enable calibration of a pulse oximetry sensor while reducing or eliminating the need to obtain such calibration information from a memory chip. An example of the calibration information is the wavelength(s) of one or more light emitting diodes. The foregoing feature may enable a reduction in the monetary cost of the oximetry sensor as compared to systems that include such a memory chip encoding the complete calibration information for the sensor. This advantage may be recognized, for example, in wireless pulse oximetry systems in which the oximetry sensor communicates with a monitor via a wireless communication protocol.

It should be noted that although presently disclosed calibration devices may, in some embodiments, eliminate the need for a memory chip, in other embodiments, the provided calibration devices may be utilized in combination with a reduced capacity memory chip or an alternative calibration method such as a resistor value which encodes the sensor type or approximate LED calibration. The reduced capacity memory device may include partial calibration information or no calibration information, but may also include other relevant information, such as information relating to providing an appropriate pulse modulated signal, indicating the type of sensor (e.g., finger, nose, etc.) or style of sensor (e.g., clip style, bandage style, etc.), the quantity of LEDs in the sensor, an identifier of the manufacturer of the sensor, and so forth. In embodiments in which the presently disclosed calibration devices are provided in combination with a reduced capacity memory device, the overall cost of the sensor or patient monitoring system may still experience reductions in monetary cost as compared to systems having a full capacity memory device due to the reduction in capacity of the memory device. Further, in some embodiments, presently disclosed calibration devices may be included in the oximetry sensor along with a reduced capacity memory chip that is provided to include encryption information, thus reducing or eliminating the likelihood that the oximetry sensor can be easily counterfeited, but providing monetary cost reductions because the need to program the memory chip with calibration information is eliminated.

Embodiments of the calibration devices disclosed herein may enable a reduction or elimination of the use of a memory chip, by providing an alternate method for obtaining calibration information relating to the wavelength of one or more light emitting components (e.g., LEDs) in the sensor. This method may be achieved in accordance with presently disclosed embodiments by exploiting features of the light emitting components. For example, in certain embodiments, the calibration information for the particular LED(s) in a given sensor may be obtained without the need for the memory chip encoding the calibration information, by obtaining the calibration information relating to the wavelength of the LED from the bandgap voltage of the LED. This feature may enable a monetary cost reduction while still providing a sensor calibration curve that corresponds to the actual wavelength(s) of the sensor's LEDs.

By measuring the forward voltage of the LED for calibration purposes, the calibration can dynamically adapt to changes in LED temperature. The environment used during the factory calibration and the patient use environment may differ. For example, the air temperature of the environment may be different or the LED drive current, duty cycle, or drive waveform may be different, resulting in slightly different heating and therefore wavelength shifts. The wavelength emitted by the LED may change with temperature and drive current as described in more detail in U.S. application Ser. No. 13/077,164, now U.S. Patent Application Publication No. 2012/0248985.

In another embodiment, a memory device with multiple calibration curves or coefficients related to temperature or operating conditions may be used. The memory device may encode a forward voltage which corresponds to each set of calibration information. During operation, the system may measure the forward voltage across one or more LEDs and then identify the corresponding calibration curve in the sensory memory. The corresponding curve may represent the closest match (due to temperature variations). Physiological parameters may then be calculated using the calibration curve which matches best with the current operating voltage of the LED.

Further, in other embodiments, a calibration element that stores multiple calibration curves or coefficients for use at different operating temperatures and/or drive currents may be provided. For example, in some instances, a different calibration curve may be suitable for a drive current of approximately 3 mA than for a drive current of approximately 50 mA because an LED wavelength shift may occur when the different drive current levels are utilized. In such cases, it may be desirable to provide a memory chip that encodes multiple calibration curves or coefficients, each corresponding to a drive current level or operating temperature, or a range of drive current levels or operating temperatures. This type of memory chip may be provided alone or in combination with presently disclosed calibration devices. For example, in one embodiment, a memory chip having different calibration curves or coefficients for drive current or operating temperature ranges may be utilized to obtain a rough approximation of the wavelength of one or more LEDs, and presently disclosed calibration devices may be utilized to fine tune this approximation during operation as the drive current or operating temperature fluctuates with device operation.

Additionally, in still further embodiments, certain systems may include a traditional calibration type resistor having a low tolerance (e.g., an approximately 1% error) and a high temperature coefficient such that measuring the resistance also gives an indication of ambient temperature. In these embodiments, the resistor may be located in the connector (ambient air) or next to the LED to better indicate die temperature. Therefore, a measurement of the resistance would give an indication of the sensor temperature and the resistor type calibration may be adjusted to reflect temperature.

In another embodiment, calibration may be accomplished by forward voltage measurement without the use of a memory device. This design may reduce the cost of the sensor for low-cost applications. In an embodiment, the LEDs are tested and grouped together by wavelength, prior to being incorporated into the sensor. For example, the purchased LEDs are tested and then sorted by wavelength into some number of bins. LEDs that differ in emitted wavelength due to slight differences in manufacturing are sorted into respective bins. A particular sensor type is manufactured with LEDs taken from only selected bins. As a result, the wavelength measured at the factory is known, and this information can be used along with the forward voltage information to provide accurate calibration when the sensor is in use. Other bins of LEDs may be used in other sensor types.

Additionally, by providing a calibration device or system configured to measure the wavelength of one or more LEDs in the oximetry sensor, periodic determination of the actual operating wavelength of the LEDs may be enabled. That is, at one or more predetermined or operator selected time points during operation, the wavelength of the LEDs may be determined and utilized to periodically recalibrate the system. For example, in some embodiments, the system may be recalibrated periodically to compensate for temperature changes of the LEDs that may cause the wavelength of the LEDs to shift during operation. In this way, in some implementations, application-specific operational factors may be accounted for during operation by recalibrating the sensor as appropriate when changes occur, thus improving the accuracy of the obtained measurements.

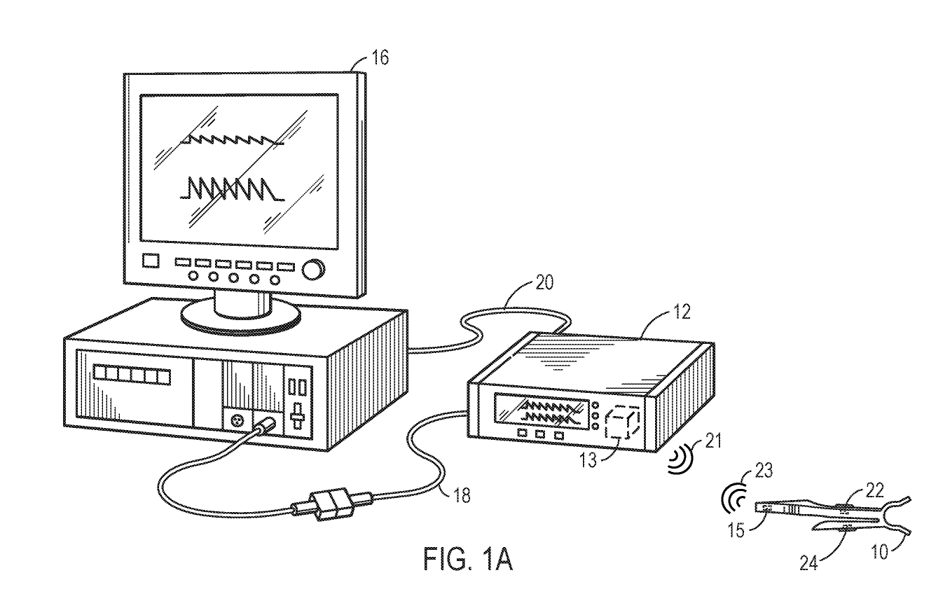

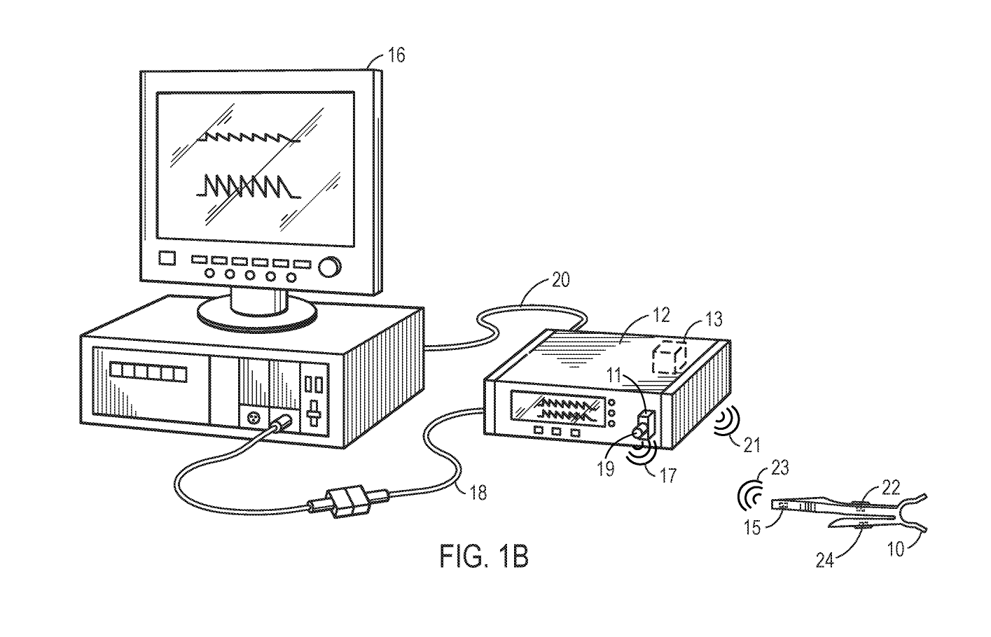

Turning now to the drawings, FIG. 1A illustrates a patient monitoring system that may utilize embodiments of the sensor calibration systems and methods described herein in the process of initializing the system for monitoring a physiological characteristic of a patient and processing the obtained data. More specifically, the illustrated system may be capable of calibrating the system, acquiring signals that correspond to detected waveforms from a sensor, and further processing the signals to extract information that may be useful in the physiological monitoring process. To that end, the following description of the patient monitoring system serves as a basis for describing the calibration techniques described in more detail below.

The patient monitoring system of FIG. 1A includes a sensor 10 and a patient monitor 12. In the embodiment illustrated in FIG. 1A, the sensor 10 and the monitor 12 are communicatively coupled via a suitable wireless communication protocol. Accordingly, the monitor 12 includes a wireless module 13 and the sensor 10 includes a wireless module 15 to facilitate transmitting and receiving of wireless data, as indicated by communication lines 21 and 23, respectively. It should be noted that the sensor 10 and the patient monitor 12 may communicate via any suitable wireless means, such as using radio, infrared, or optical signals. Accordingly, the wireless modules 13 and 15 may be any suitable type of transmission devices capable of facilitating wireless transmission between the sensor 10 and the patient monitor 12.

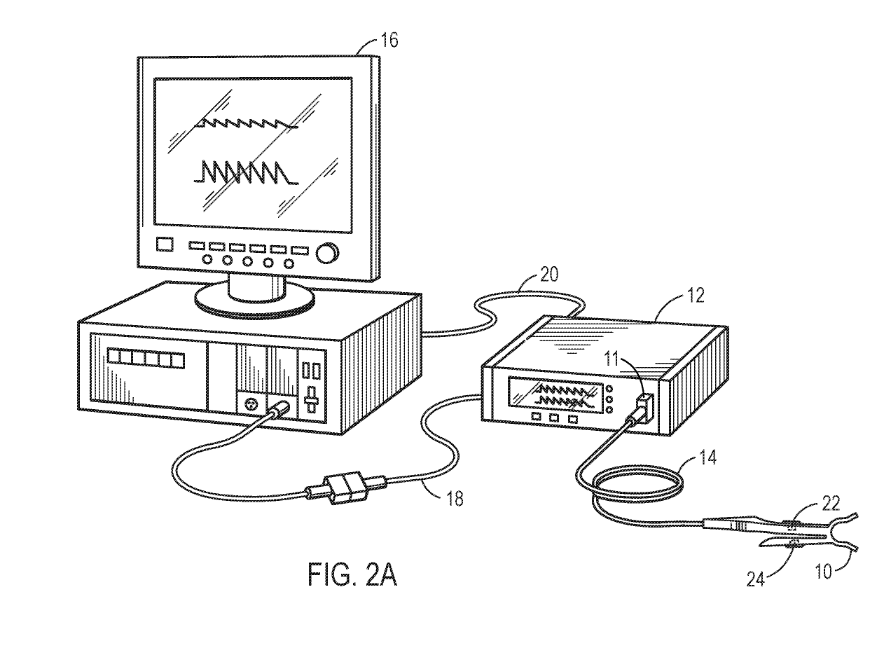

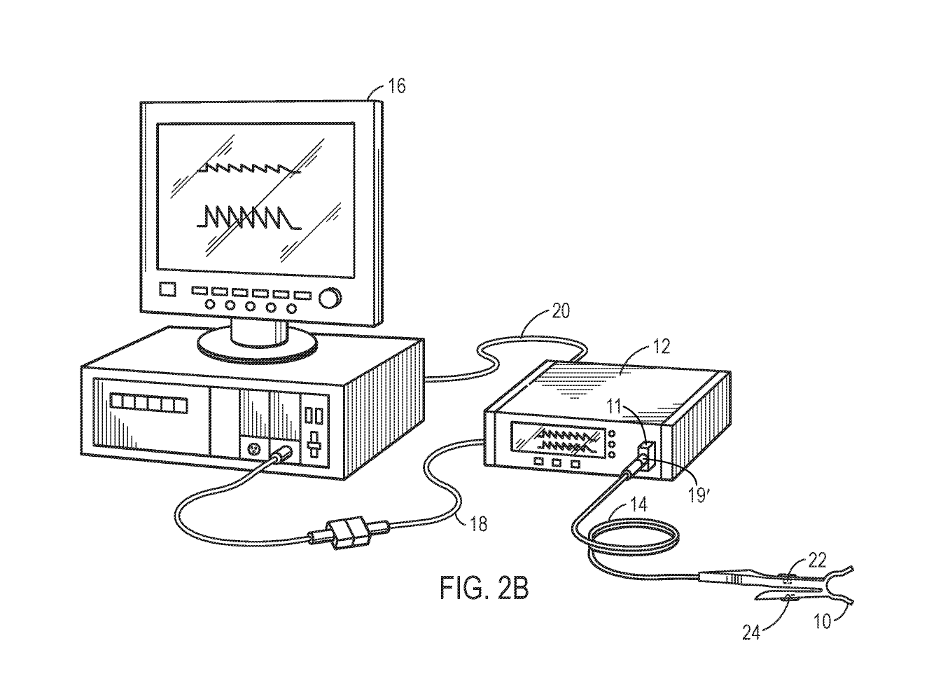

However, it should be noted that the presently disclosed calibration systems and methods may be utilized with wireless patient monitoring systems, such as the systems shown in FIGS. 1A and 1B, with wired patient monitoring systems, such as the systems shown in FIGS. 2A and 2B, or with a combination of wired and wireless monitoring systems. In the wired embodiment of FIG. 2A, a cable 14 connects the sensor 10 to the patient monitor 12 to enable the exchange of data between the sensor 10 and the monitor 12. In this embodiment, the wireless modules 13 and 15 may be eliminated if wireless data communication is not desired as an option. However, it should be noted that presently disclosed calibration systems and methods may be utilized in combination with patient monitoring systems that utilize wireless communication, wired communication, or a combination thereof. Indeed, the communication protocol(s) and device(s) that communicatively couple the components of the system may be chosen based on implementation-specific considerations.

As will be appreciated by those of ordinary skill in the art, the sensor 10 and/or the cable 14, if utilized in the given implementation, may include or incorporate one or more integrated circuit devices or electrical devices, such as a memory, processor chip, or resistor, that may facilitate or enhance communication between the sensor 10 and the patient monitor 12. Likewise, in embodiments that include the cable 14, the cable 14 may be an adaptor cable, with or without an integrated circuit or electrical device, for facilitating communication between the sensor 10 and various types of monitors, including older or newer versions of the patient monitor 12 or other physiological monitors. As will be appreciated by those of ordinary skill in the art, the cable 14 (or corresponding wireless transmissions) are typically used to transmit control or timing signals from the monitor 12 to the sensor 10 and/or to transmit acquired data from the sensor 10 to the monitor 12. In some wired embodiments, however, the cable 14 may be an optical fiber that allows optical signals to be conducted between the monitor 12 and the sensor 10.

In one embodiment, the patient monitor 12 may be a suitable pulse oximeter, such as those available from Nellcor Puritan Bennett LLC. In other embodiments, the patient monitor 12 may be a monitor suitable for measuring tissue water fractions, or other body fluid related metrics, using spectrophotometric or other techniques. Furthermore, the monitor 12 may be a multi-purpose monitor suitable for performing pulse oximetry and measurement of tissue water fraction, or other combinations of physiological and/or biochemical monitoring processes, using data acquired via the sensor 10. Furthermore, to upgrade conventional monitoring functions provided by the monitor 12 to provide additional functions, the patient monitor 12 may be coupled to a multi-parameter patient monitor 16 via a cable 18 connected to a sensor input port and/or via a cable 20 connected to a digital communication port.

In the example shown in FIG. 1A, the sensor 10 is a clip-style sensor including an emitter 22 and a detector 24 which may be of any suitable type. For example, the emitter 22 may be one or more light emitting diodes (LEDs) capable of transmitting one or more wavelengths of light, such as in the red to infrared range, and the detector 24 may be a photodetector, such as a silicon photodiode package, selected to receive light in the range emitted from the emitter 22. In the illustrated embodiment, the sensor 10 is coupled to the cable 14 that is responsible for transmitting electrical and/or optical signals to and from the emitter 22 and detector 24 of the sensor 10. The cable 14 may be permanently or removably coupled to the sensor 10, depending on features of the implementation. For example, in instances in which the sensor 10 is disposable, the cable 14 may be removably coupled, for example, for cost efficiency purposes.

The sensor 10 described above is generally configured for use as a “transmission type” sensor for use in spectrophotometric applications, though in some embodiments it may instead be configured for use as a “reflectance type sensor.” Further, in other embodiments, the sensor 10 may be any suitable oximeter associated with an embodiment of the presently disclosed calibration systems. For example, the sensor 10 may be an in-vivo optical spectroscopy oximeter capable of measuring changes in oxygen levels of a patient. Indeed, the sensor 10 may be any of a variety of types of light emitting sensors employed by those skilled in the art, not limited to the particular types of sensors that are described in detail herein.

Transmission type sensors include an emitter 28 and detector 32 that are typically placed on opposing sides of the sensor site. If the sensor site is a fingertip, for example, the sensor 10 is positioned over the patient's fingertip such that the emitter 28 and detector 32 lie on either side of the patient's nail bed. For example, the sensor 10 is positioned so that the emitter 28 is located on the patient's fingernail and the detector 32 is located opposite the emitter 28 on the patient's finger pad. During operation, the emitter 28 shines one or more wavelengths of light through the patient's fingertip, or other tissue, and the light received by the detector 32 is processed to determine various physiological characteristics of the patient.

Reflectance type sensors generally operate under the same general principles as transmittance type sensors. However, reflectance type sensors include an emitter and detector that are typically placed on the same side of the sensor site. For example, a reflectance type sensor may be placed on a patient's fingertip such that the emitter and detector are positioned side-by-side. Reflectance type sensors detect light photons that are scattered back to the detector.

For pulse oximetry applications using either transmission or reflectance type sensors, the oxygen saturation of the patient's arterial blood may be determined using two or more wavelengths of light, most commonly red and near infrared wavelengths. Similarly, in other applications, a tissue water fraction (or other body fluid related metric) or a concentration of one or more biochemical components in an aqueous environment may be measured using two or more wavelengths of light, most commonly near infrared wavelengths between about 1,000 nm and about 2,500 nm. It should be understood that, as used herein, the term “light” may refer to one or more of infrared, visible, ultraviolet, or even X-ray electromagnetic radiation, and may also include any wavelength within the infrared, visible, ultraviolet, or X-ray spectra.

Pulse oximetry and other spectrophotometric sensors, whether transmission-type or reflectance-type, are typically placed on a patient in a location conducive to measurement of the desired physiological parameters. For example, pulse oximetry sensors are typically placed on a patient in a location that is normally perfused with arterial blood to facilitate measurement of the desired blood characteristics, such as arterial oxygen saturation measurement (SpO₂). In such a system, generally, the light generated by the emitter 22 and passed through the patient's tissue is selected to be of one or more wavelengths that are absorbed by the blood in an amount representative of the amount of the blood constituent present in the blood. The amount of blood passed through the tissue varies in accordance with the changing amount of blood constituent and the related light absorption.

In certain embodiments, the emitter 22 may emit at least two (e.g., red and infrared (IR)) wavelengths of light. The red wavelength may be between about 600 nm and about 700 nm (e.g., a red light emitted at about 660 nm), and the IR wavelength may be between about 800 nm and about 1000 nm (e.g., a near-infrared light emitted at about 900 nm). It should be noted, however, that any appropriate wavelength (e.g., green, yellow, etc.) and/or any number of wavelengths (e.g., one, two, three or more) may be used in other embodiments. In embodiments in which blood oxygen saturation is desired, the ratio-of-ratios (Ratrat) of the absorption of red light to near-infrared light may be calculated. The AC and DC components of the RED wavelength and IR wavelength signals generated by passing light through the patient are typically used to calculate Ratrat, which has been observed to correlate well to blood oxygen saturation.

Accordingly, pulse oximeters, such as those shown in FIGS. 1A and 2A, may measure Ratrat in order to determine blood oxygen saturation. The relationship between Ratrat and blood oxygen saturation follows a line that serves as a calibration curve for the sensor 10. Because the light absorption of the blood's oxygenated hemoglobin and deoxygenated hemoglobin is wavelength-dependent, the relationship between Ratrat and blood oxygen saturation may depend upon the specific wavelength emitted by the sensor's light emitting diodes (LEDs). Accordingly, the accuracy of the blood oxygen saturation measurements obtained with the pulse oximetry system may depend on use of a calibration curve that corresponds to the actual wavelengths of the sensor's LEDs. Embodiments of the presently disclosed calibration systems and methods may reduce or eliminate the likelihood of the sensor's calibration curve being shifted, rotated, or otherwise distorted due to improper calibration of the device while also reducing or eliminating the need for a digital memory device encoding the LED wavelength.

In certain embodiments, the digital memory device may be eliminated from the sensor 10 or may have reduced functionality, but the monitor 12 may recognize the sensor 10 to be of the conventional type having a full memory chip. To that end, in certain embodiments, as shown in FIGS. 1B and 2B, a sensor 10 having a reduced capacity memory chip or no memory chip may be provided with a wireless dongle 19 or a wired dongle 19′ to manipulate the monitor 12 to function as if coupled to a traditional sensor 10. That is, the dongles 19 and 19′ may be configured to gather the calibration information from the provided calibration devices along with any information provided by the reduced capacity memory chip and to provide such information to the controller in the monitor 12. In this way, the monitor 12 may receive the calibration and other stored information as if a traditional sensor was wirelessly coupled or wired to the monitor 12.

The foregoing feature may enable the wired and wireless systems of FIGS. 1A and 2A to be retrofitted, such that an existing monitor 12 may function with the sensor 10 without reconfiguring the monitor 12. Specifically, in the wireless embodiment shown in FIG. 1B, the dongle 19 is coupled to the sensor cable connection port 11 and is configured to wirelessly communicate with the sensor 10 and/or controller 13, as illustrated by communication lines 17. In this way, the dongle 19 may interrupt or override the monitor-to-sensor and/or sensor-to-monitor communication stream to manipulate the monitor 12 such that the monitor 12 does not recognize that a modified sensor 10 is being utilized. This feature enables a user to utilize the sensor 10 with an existing monitor 12, for example, by plugging the dongle 19 into the monitor 12 before initiating operation of the system. Although the dongle 19 is shown coupled to the sensor cable connection port 11 in FIG. 1B, in other embodiments, the dongle 19 may be connected to any suitable port in the monitor 12, such as an existing port, a multi-pin connection, or any other provided port. Further, in some embodiments, the dongle 19 may be configured to couple to the multi-parameter monitor 16, the monitor 12, or both.

In the wired embodiment shown in FIG. 2B, the dongle 19′ is coupled to the sensor cable connection port 11, and the sensor cable 14 is plugged into the dongle 19′. That is, in certain embodiments, a user may utilize the dongle 19′ with the sensor 10 having a presently disclosed calibration device to manipulate the monitor 12 into recognizing the sensor 10 and dongle 19′ as a traditional sensor. The foregoing feature may enable existing monitors 12 and 16 to be retrofit with the sensors having the calibration devices disclosed herein.

For example, in the case where the sensor expects the sensor to contain a one-wire non-volatile memory device, the dongle implementation may contain a micro-controller which determines the sensor calibration through a wired or wireless connection and then emulates the functionality of the one wire non-volatile memory by providing the monitor with calibration data in the expected format that corresponds to the measured values. Other interfaces such as a universal asynchronous receiver/transmitter and/or a serial peripheral interface bus are also suitable in this application. The micro-controller may be replaced by any other logic or logic device, such as a compact programmable logic device or a field programmable gate array.

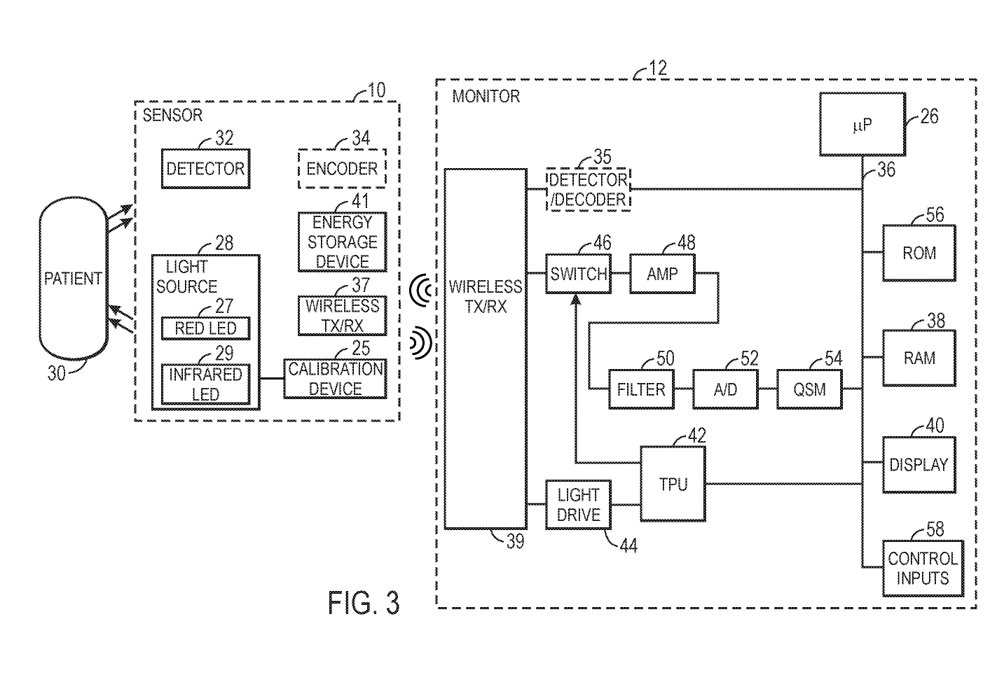

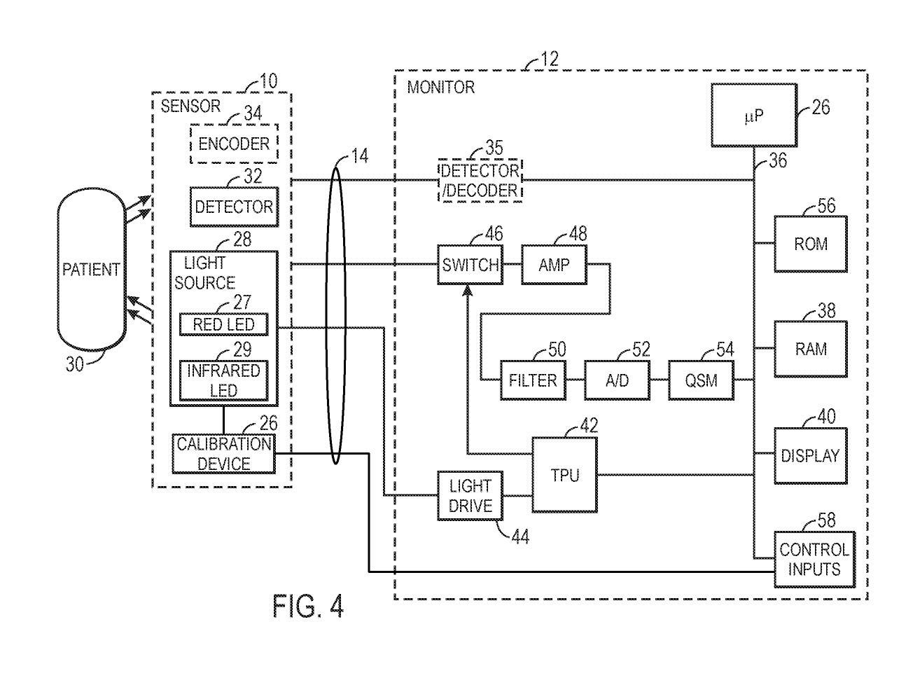

FIG. 3 is a block diagram of an embodiment in which the patient monitor is a pulse oximeter 12 and the sensor 10 is an oximetry sensor that includes a calibration device 25 capable of implementing embodiments of presently disclosed sensor calibration methods. In this embodiment, the monitor 12 and the sensor 10 are wirelessly coupled, for example, as shown in FIG. 1. However, FIG. 4 is a block diagram of an embodiment in which the pulse oximeter 12 and the sensor 10 are coupled via cable 14, for example, as shown in FIG. 2. Again, the sensor 10 and the monitor 12 may be communicatively coupled in any desired manner.

Various embodiments of the presently disclosed calibration methods may be implemented in whole or in part in a calibration device 25 located, for example, in a body of the sensor 10. The obtained calibration information (or raw data from which calibration information, such as calibration coefficients, may be obtained) may be utilized in one or more data processing algorithms that are executed by a microprocessor 26, which is provided as a component of the pulse oximeter 12 in the illustrated embodiments. Further, it should be noted that the embodiments of the present invention may be implemented as a part of a larger signal processing system used to process signals for the purpose of determining a desired physiological characteristic. As such, the microprocessor 26 may be operated alone or in conjunction with other processors in the signal processing system to implement the presently disclosed calibration and signal processing methods.

Turning now to operation of the illustrated systems, light from a light source 28 passes into a blood perfused tissue of a patient 30 and is scattered and detected by photodetector 32 (or any other suitable light detecting element). The light source 28 includes one or more light emitting elements, which are depicted as a red LED 27 and an infrared LED 29 in FIGS. 3 and 4. In some embodiments, the sensor 10 containing the light source 28 and the photodetector 32 may also contain an encoder 34 that provides signals indicative of information about the light source 28 to a decoder 35 to enable the pulse oximeter 12 to properly control operation of the sensor 10. In some embodiments, the encoder 34 may, for example, be a memory device.

However, in certain embodiments, the encoder 34 may not be present, and the calibration device 26 may provide the monitor 12 signals indicative of the wavelengths of the LEDs 27 and 29 to enable the pulse oximeter 12 to select appropriate calibration coefficients for calculating oxygen saturation. That is, the calibration device 26 may reduce or eliminate the need for the encoder 34, thus reducing monetary cost of the sensor 10. It should be noted, however, that in some embodiments, the encoder or other memory device may be included, but may have reduced capacity. For example, in certain embodiments, the encoder 34 may not include the wavelength of the LEDs 27 and 29, but may include information about the sensor 10, such as information relating to providing an appropriate pulse modulated signal, indicating the type of sensor (e.g., finger, nose, etc.) or style of sensor (e.g., clip style, bandage style, etc.), the quantity of LEDs in the sensor, an identifier of the manufacturer of the sensor, and so forth.

For further example, in some embodiments, the encoder 34 may not include calibration information, thus reducing monetary cost of the sensor 10, but may include encrypted data for the purpose of reducing or preventing counterfeit sensors. The encrypted data may be utilized, for example, to enable the monitor, or monitors with which the sensor is configured to work, to recognize the sensor 10 as a legitimate device for operation. In this way, sensors that do not include the necessary encrypted data may not be functional when coupled to monitors that are configured to check for the presence of the encrypted data. Such encryption enhances patient safety by preventing the use of sensors that may not be properly calibrated.

The sensor 10 is connected to the pulse oximeter 12 either via cable 14, as in the embodiment of FIG. 4, or via wireless transmitters 37 and 39, as shown in FIG. 3. In certain wired embodiments, the sensor 10 may derive power from the monitor 12. However, in some wireless embodiments, the sensor 10 may include or may be coupled to an energy storage device 41 to supply the sensor array 14 with power. By way of example only, the energy storage device 41 may, in some embodiments be a battery, which may be a rechargeable battery (e.g., a lithium ion, lithium polymer, nickel-metal hydride, or nickel-cadmium battery) or a single-use battery such as an alkaline or lithium battery.

As shown, the pulse oximeter 12 includes the microprocessor 26 connected to an internal bus 36. A random access memory (RAM) memory 38 and a display 40 are also connected to the bus 36. A time processing unit (TPU) 42 provides timing control signals to light drive circuitry 44, which controls when light source 28 is illuminated and, if multiple light sources are used, the multiplexed timing for the different light sources. The TPU 42 also controls the gating-in of signals from photodetector 32 through a switching circuit 46. These signals are sampled at the proper time, depending upon which of multiple light sources is illuminated, if multiple light sources are used. The received signal is passed through an amplifier 48, a low pass filter 50, and an analog-to-digital converter 52. The digital data is then stored in a queued serial module (QSM) 54, for later downloading to RAM 38 as QSM 54 approaching its capacity. In one embodiment, there may be multiple parallel paths of separate amplifier, filter and A/D converters for multiple light wavelengths or spectra received.

Based on the value of the received signals corresponding to the light received by photodetector 32, microprocessor 26 will calculate the desired blood characteristics, such as blood oxygen saturation, using various algorithms. These algorithms may require coefficients, which may be empirically determined, corresponding to, for example, the wavelengths of light used by the light source 28 and determined via the calibration device 25. These and other parameters, constants, and so forth, may be stored in a read only memory (ROM) 56. In a two-wavelength system, the particular set of coefficients chosen for any pair of wavelength spectra is determined by the values indicated by the calibration device 25 corresponding to a particular light source in a particular sensor 10. Additionally, a variety of control inputs 58 may be utilized in the calculation of the desired blood characteristics. Control inputs 58 may be, for instance, a switch on the pulse oximeter, a keyboard, or a port providing instructions from a remote host computer. Furthermore, any number of methods or algorithms may be used to determine a patient's pulse rate, oxygen saturation or any other desired physiological parameter.

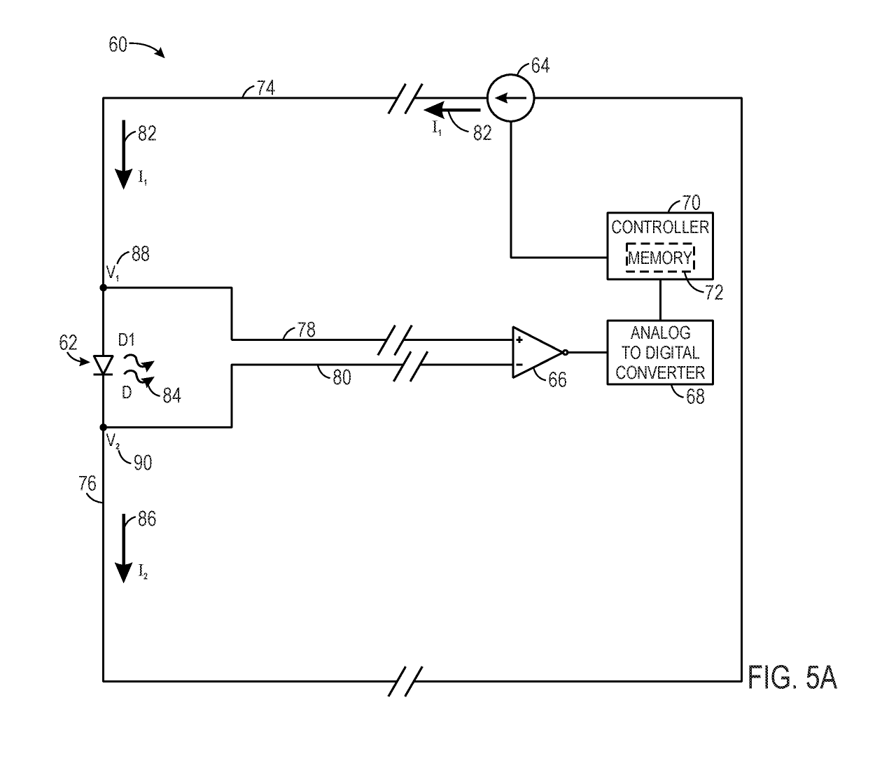

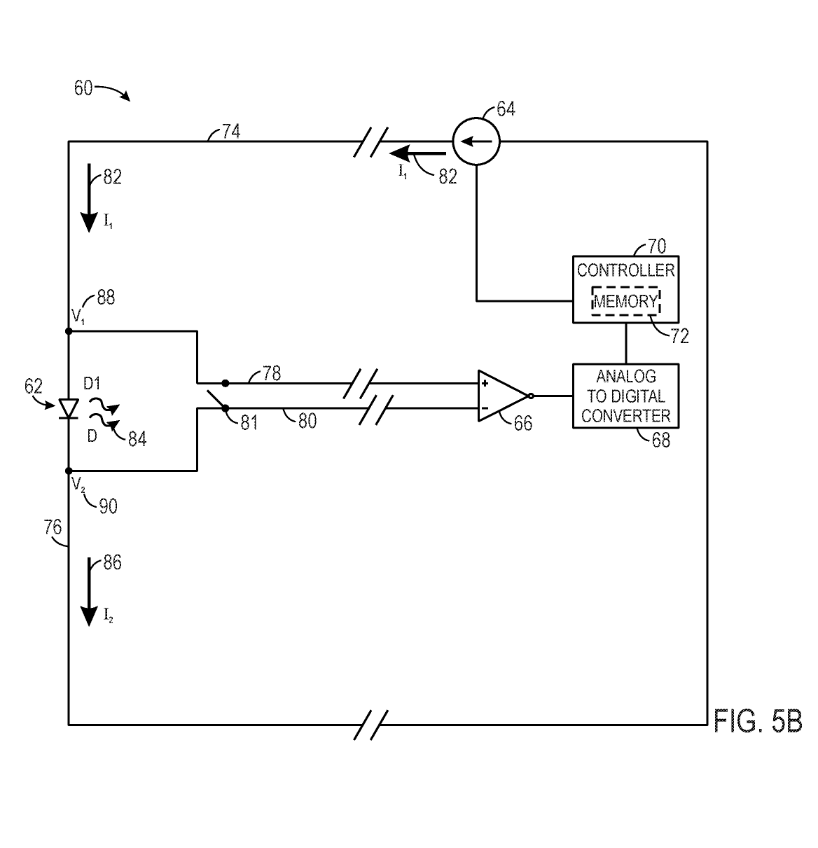

FIG. 5A depicts an embodiment of a calibration circuit 60 that may be included in some embodiments of the calibration device 25. The calibration circuit 60 is configured such that during operation, calibration data containing a wavelength of an LED 62 may be empirically determined by exploiting the fact that the desired calibration information is effectively stored in the bandgap voltage of the LED 62. For example, the bandgap voltage (i.e., the voltage at which the electrons in a current passed through the LED cross from the valence band to the conduction band) for the LED is given by:

V=(hc)/λ, (1)

where V is the bandgap voltage, h is Planck's constant, c is the speed of light, and λ is the wavelength of the LED. This bandgap relationship may be exploited to determine the wavelength of the LED 62 without the need for a memory chip (e.g., encoder 34). That is, by passing a current through the LED 62 and measuring the bandgap voltage, the wavelength of the LED 62 may be calculated without the need for a memory chip that provides the LED wavelength.

More specifically, in one embodiment, the calibration circuit 60 may be operated to experimentally obtain the wavelength of the LED 62. To that end, as shown in FIG. 5A, the calibration circuit 60 includes a current source 64, a high impedance amplifier 66, an analog to digital converter 68, and a controller 70 having memory 72. It should be noted that in some embodiments, certain portions of the calibration circuit 60 may be located in a cable (e.g., a cable having multiple conductors or wires for electrical coupling of components of the circuit) while other portions of the calibration circuit 60 may be located on a circuit board. For example, in one embodiment, lines 74, 76, 78, and 80 may represent wires or conductors that couple together elements of the calibration circuit 60. These wires or conductors may be combined into a cable in some embodiments. Further, in one embodiment, the amplifier 66, analog to digital converter 68, and controller 70 may be located on a circuit board coupled to the cable containing the wiring. However, in other embodiments, the components of the circuit may be coupled together in any of a variety of desired ways, depending on implementation-specific considerations.

In the illustrated embodiment, the line 74 represents a connection between an electrical output of the current source 64 and an electrical input of the LED 62. Similarly, the line 76 represents a connection between the electrical output of the LED 62 and the electrical input of the current source 64. Further, the line 78 represents a voltage sensing lead coupled to the electrical input of the LED 62, and the line 80 represents a voltage sensing lead coupled to the electrical output of the LED 62. Again, it should be noted that in some embodiments, each of these lines 74, 76, 78, and 80 may represent separate wires, cables, or conductors coupled together in the illustrated manner. As described in more detail below, by providing the connections 78 and 80 (in addition to the connections 74 and 76) for the purpose of sensing the voltage at the input and output of the LED 62, respectively, and utilizing a high impedance amplifier 66 to amplify the sensed voltages, the measurement error due to the relatively high resistance of the cables 74 and 76 compared to the resistance of the LED 62 may be reduced or eliminated.

The described components of the circuit 60 may be utilized to obtain calibration information relevant to operation of the sensor and processing of data obtained during use of the sensor. For example, the circuit 60 may be utilized to obtain the forward voltage of the LED 62, which may be subsequently used to determine a calibration coefficient and/or curve for the sensor. To that end, during operation of the circuit 60, the controller 70 outputs a control signal that directs the current source 64 to output a current 82 to the LED 62 via connection 74. The current 82 passes through the LED 62 causing the LED 62 to produce light, as indicated by arrows 84, and a second current 86 leaves the LED 62.

During this process, a voltage drop across the LED 62 is measured by voltage sensing leads 78 and 80. More specifically, the voltage sensing lead 78 senses a first voltage 88 present at the electrical input of the LED 62, and the voltage sensing lead 80 senses a second voltage 90 present at the electrical output of the LED 62. The sensed voltages 88 and 90 are passed to the amplifier 66. The amplifier 66 amplifies the received signals and provides an output to the analog to digital converter 68, which converts the output to a digital signal and transmits the digital signal to the controller 70.

Referring again to FIG. 5A, the circuit 60 may be operated without the wires 78 and 80. The measured voltage drop would also include the resistance of the cable; however, if the cable is short or the resistance is known, then the wires 78 and 80 may be omitted.

In one embodiment, the digital signal is then processed by the controller 70 to determine a difference between the first voltage 88 and the second voltage 90. This voltage difference represents the voltage drop across the LED 62, which corresponds to the forward voltage of the LED 62. The controller 70 utilizes equation (1), which may be stored, for example in memory 72, to calculate the wavelength of the LED 62. The controller 70 then determines calibration information for the sensor, such as a calibration coefficient or curve, based on the experimentally determined wavelength of the LED 62, and communicates this information via wireless or wired transmission to the processor 26 for use in calibrating the sensor and processing signals acquired with the sensor. This may be performed at startup to initially calibrate the LED 62 and/or periodically during operation, for example, to compensate for the temperature drift of the LED 62. The time points at which calibration information is obtained may be preset, set by an operator, determined by a controller, or chosen based on any other implementation-specific factor. It should further be noted that in other embodiments, the controller 70 function may be more limited than previously described, and some or all of the processing of the sensed voltages 88 and 90 and/or control of one or more switches that are periodically activated to obtain calibration information may be performed by another controller or set of controllers (e.g., processor 26).

In some embodiments, certain features and modes of operating the circuit 60 may enable efficient acquisition of the forward voltage of the LED 62 while reducing noise. For example, in one embodiment, the level of the current 82 may be selected such that the current level corresponds to a minimum current (e.g., approximately 1 mA) necessary to cause the LED 62 to emit light. In this embodiment, the level of the current 82 may be chosen in this manner so that a voltage error created by the internal resistance of the LED 62 may be reduced, minimized, or eliminated. Further, in certain embodiments, by utilizing voltage sensing leads 78 and 80 to measure the voltages 88 and 90, the impact of the resistance introduced by the current carrying wires on the forward voltage calculation may be reduced or eliminated. For example, in one embodiment, the dimensions and specifications of the voltage sensing leads 78 and 80 may be chosen such that the resistance of the leads 78 and 80 is small compared to the high impedance of the amplifier 66.

It should be noted that although the circuit 60 of FIG. 5A illustrates one LED 62 and circuitry suitable for determining the wavelength of one LED, in other embodiments, the circuit 60 may be adapted to determine the wavelengths of any quantity of LEDs. For example, in one embodiment, the sensor 10 may include the red LED 27 and the infrared LED 29, and the circuit 60 may be adapted to determine a first wavelength of the red LED 27 and a second wavelength of the infrared LED 29. In such an embodiment, each LED may have a dedicated circuit, or a combined circuit may include and measure values associated with both LEDs.

Further, in some implementations, it may be desirable to selectively couple and decouple certain portions of the circuit 60 from the active circuit path. For example, in some embodiments, it may be desirable to couple the amplifier 66 and the leads 78 and 80 to the active circuit path during testing of the LED wavelength but to decouple such components from the circuit during normal operation to reduce the resistance of the main lines. To that end, one or more switches, switching devices, or switching controllers may be included in some embodiments of the circuit 60. For example, as shown in FIG. 5B, a switch 81 may be provided to couple and decouple the wavelength determining circuit components from the active circuit path. In the illustrated embodiment, the controller 70 opens the switch 81 to determine the LED wavelength and closes the switch 81 when the sensor is utilized on the patient for measurement acquisition. However, in other embodiments, any quantity of switches may be provided in the circuit 60 to selectively isolate certain portions of the circuit during different periods of use.

In certain embodiments described herein, the calibration circuit 60 is utilized to measure the forward voltage drop across LED 62, and the forward voltage drop is utilized to obtain the wavelength of the LED 62, for example, by utilizing equation (1). However, in other embodiments, the circuit 60 may be operated to measure the forward voltage, and the forward voltage may be utilized to directly obtain calibration information without the need to calculate the wavelength of the LED 62. In such embodiments, known information about the LED 62 (e.g., process used to manufacture the LED, identity of the manufacturer of the LED, etc.) may be utilized to bypass the wavelength calculation and directly correlate the measured forward voltage to a calibration coefficient or curve.

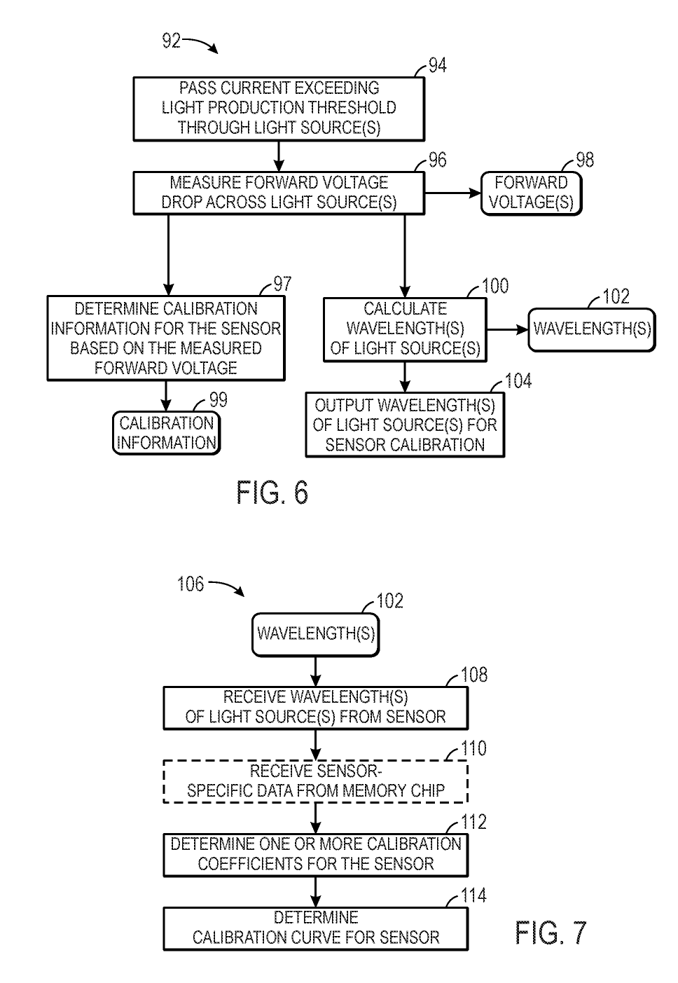

FIG. 6 is a flow chart illustrating an embodiment of a method 92 for experimentally determining calibration information and/or the wavelength(s) of one or more light sources. As illustrated, the method 92 includes passing current through one or more light sources at a level that exceeds each light source's light production threshold (block 94). The forward voltage drop across each light source is then measured (block 96) to determine the forward voltages 98 of the light sources. In one embodiment, the forward voltages 98 may then be utilized in equation (1) to calculate the wavelengths 102 of the light sources (block 100). The wavelengths 102 of the light sources are then output for use in calibration of the sensor (block 92).

In another embodiment, once the forward voltages 98 are measured, calibration information 99 is then determined for the sensor based at least in part on the measured forward voltages 98 (block 97). For example, the calibration information 99 may include but is not limited to a calibration coefficient or calibration curve for the sensor. In such embodiments, the forward voltages 98 may be used either lone or in combination with other known or acquired information to determine the calibration information 99. For instance, other factors such as the sensor manufacturer, the manufacturing process used to make the sensor, additional calibration information encoded by an encoder, operating temperature, drive current, and so forth, may also be taken into account along with the measured forward voltages 98 to determine the calibration information 99. Further, it should be noted that in some embodiments, all or some of the steps of the method 92 may be performed by the controller 70 local to the circuit 60.

FIG. 7 is a flow chart illustrating an embodiment of a method 106 for utilizing an experimentally determined light source wavelength for calibration of a sensor containing the light source. In this embodiment, the wavelength 102 of the light source is received from the sensor (block 108). For example, the processor 26 may receive the experimentally determined wavelength 102 from the controller 70 in the sensor 10. Additionally, in some embodiments, sensor-specific data may be received from a memory chip (e.g., encoder 34) in the sensor. For example, information may be received that relates to providing an appropriate pulse modulated signal, indicating the type of sensor (e.g., finger, nose, etc.) or style of sensor (e.g., clip style, bandage style, etc.), the quantity of LEDs in the sensor, an identifier of the manufacturer of the sensor, and so forth. The method 106 also calls for determining one or more calibration coefficients and/or calibration curves for the sensor based on the received wavelength(s) (blocks 112 and 114).

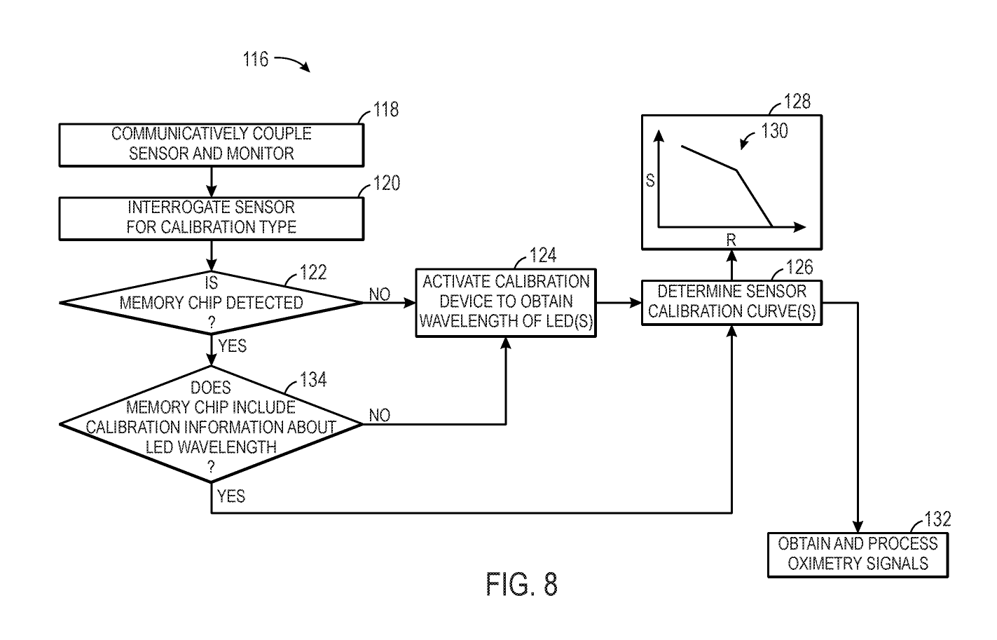

FIG. 8 illustrates an embodiment of a method 116 for initiating and calibrating a patient monitoring system. In this embodiment, the method 116 includes communicatively coupling a sensor and a monitor, for example, in a pulse oximetry system (block 118). As mentioned above, the sensor and monitor may be communicatively coupled via a wired or wireless protocol. Once coupled, the monitor interrogates the sensor for the calibration type of the sensor (block 120). For example, the monitor may interrogate the sensor to determine if the sensor has a memory chip, a calibration device, or both.

In the illustrated embodiment, the method includes an inquiry as to whether a memory chip is detected (block 122). If a memory chip is not detected, the calibration device is activated to experimentally determine the wavelengths of the one or more LEDs in the sensor (block 124). Once the wavelengths have been determined, one or more calibration curves or coefficients are determined for the sensor (block 126). For example, in the embodiment illustrated in FIG. 8, a calibration curve 128 is determined for the sensor. By way of example only, the calibration curve 128 corresponds to a plot 130 of the ratio-of-ratios (R) of the absorption of red light to near-infrared light to the oxygen saturation value (S). This curve 130 will enable the pulse oximeter to determine which oxygen saturation value to report for a given patient measurement when the pulse oximeter subsequently obtains data (block 132).

Alternatively, if a memory chip is detected during inquiry 122, the method 116 further inquires whether the memory chip includes calibration information relating to the sensor (block 134). If the memory chip is present in the sensor but does not include calibration information, the method 116 proceeds as before to utilize the calibration device to obtain the needed calibration coefficients and/or curves. However, if the memory chip does include calibration information, then the method 116 calls for this information to be accessed and utilized before obtaining and processing oximetry signals (block 132).

Further, it should be noted that in certain embodiments, the calibration devices and systems provided herein may be utilized to retrofit existing sensors of current pulse oximetry systems, or to retrofit a sensor that is manufactured without a memory chip and without a calibration device. For example, in one embodiment, sensor 10 may be modified to include the calibration device 26. Further, existing pulse oximetry systems, or other medical device systems may be updated with the calibration devices and systems provided herein.

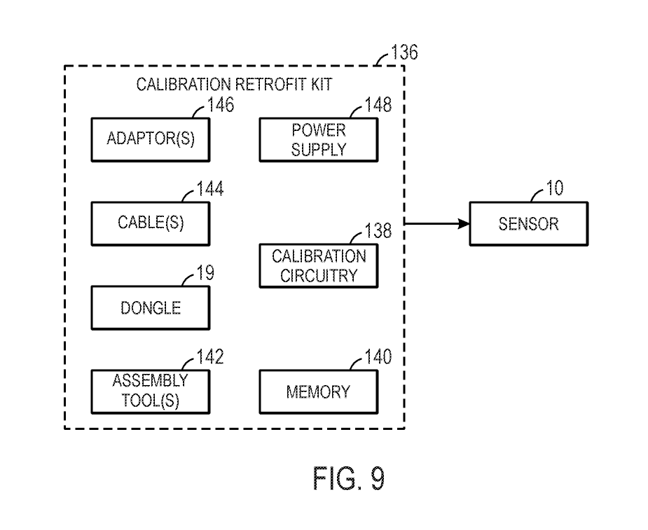

FIG. 9 is a schematic illustrating one embodiment of a calibration retrofit kit 136 that may be utilized to retrofit sensor 10, which may or may not include a calibration or memory system or device. The illustrated retrofit kit 136 includes sample components that may be included in a retrofit kit, but are not meant to limit presently contemplated embodiments. Indeed, any quantity of combination of the illustrated items may be included in the calibration retrofit kit 136 depending on implementation-specific considerations.

The depicted embodiment of the retrofit kit 136 includes dongle 19, calibration circuitry 138, memory 140, assembly tool(s) 142, cable(s) 144, adaptor(s) 146, and a power supply 148. The calibration circuitry 138 may include, for example, a printed circuit board having an amplifier, control circuitry, etc. The power supply 148 may be a current source suitable for producing the level of current desired to probe the light emitting components in the sensor 10. The adaptors 146, cables 144, and/or assembly tools 142 may be provided to enable an operator to electrically and/or physically couple the components of the retrofit kit 136 to the sensor 10. For example, in one embodiment, an adaptor 146 may be provided for coupling the sensor 10 and/or the sensor cable 14 to the dongle 19. Again, it should be noted that the illustrated components of the retrofit kit 136 are merely examples.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A wireless photoplethysmography system, comprising:
a wireless photoplethysmography sensor, comprising:
a sensor body, comprising:
an emitter configured to emit light;
a detector configured to receive the light emitted by the emitter and to generate a signal based on the light received from the emitter;
a memory storing a plurality of calibration coefficients or calibration curves corresponding to respective wavelengths of light that may be emitted by the emitter;
a calibration circuit coupled to an electrical input of the emitter to measure a first voltage present at the electrical input and coupled to an electrical output of the emitter to measure a second voltage present at the electrical output;
a processor configured to determine a voltage difference between the first voltage and the second voltage when a current is passed through the emitter, and to select an appropriate calibration coefficient or calibration curve for the wireless photoplethysmography sensor from the plurality of calibration coefficients or calibration curves stored on the memory based on the voltage difference; and
a wireless transmitter configured to wirelessly transmit the signal and the appropriate calibration coefficient or calibration curve to a monitor, wherein the appropriate calibration coefficient or calibration curve enables the monitor to calculate a physiological parameter based on the signal.

2. The system of claim 1, wherein the sensor body comprises a current source configured to generate the current passed through the emitter.

3. The system of claim 2, wherein the processor is configured to periodically determine the voltage difference and to select the appropriate calibration coefficient or calibration curve for the wireless photoplethysmography sensor during a monitoring session of a patient.

4. The system of claim 2, wherein the processor is configured to cause the current source to generate the current, to determine the voltage difference, and to select the appropriate calibration coefficient or calibration curve during an initialization process in response to wirelessly coupling the wireless photoplethysmography sensor to the monitor.

5. The system of claim 1, wherein the processor is configured to determine a wavelength of light emitted by the emitter based on the voltage difference, and to select the appropriate calibration coefficient or calibration curve based on the wavelength.

6. The system of claim 1, wherein the processor is configured to select the appropriate calibration coefficient or the calibration curve by directly correlating a value of the voltage difference to the plurality of calibration coefficients or calibration curves stored on the memory and without calculating a wavelength of light emitted by the emitter.

7. The system of claim 1, wherein the calibration circuit comprises a switch configured to move between an open position which enables the processor to determine the voltage difference and a closed position which blocks transmission of a signal indicative of the first voltage and the second voltage to the processor.

8. The system of claim 1, comprising the monitor, wherein the monitor comprises a display configured to display the physiological parameter.

9. The system of claim 1, wherein the emitter comprises a red light emitting diode configured to emit light between 600 and 700 nanometers and an infrared light emitting diode configured to emit light between 800 and 1000 nanometers.

10. A wireless photoplethysmography system, comprising:
a monitor comprising a monitor processor;
a wireless photoplethysmography sensor, comprising:
a sensor body, comprising:
an emitter configured to emit light;
a memory storing a plurality of calibration coefficients or calibration curves each corresponding to respective wavelengths of light that may be emitted by the emitter;
a calibration circuit coupled to an electrical input of the emitter to measure a first voltage present at the electrical input and coupled to an electrical output of the emitter to measure a second voltage present at the electrical output;
a sensor processor configured to determine a voltage difference between the first voltage and the second voltage when a current is passed through the emitter, and to select an appropriate calibration coefficient or calibration curve for the photoplethysmography sensor from the plurality of calibration coefficients or calibration curves stored on the memory based on the voltage difference; and
a wireless transmitter configured to wirelessly transmit the appropriate calibration coefficient or calibration curve to the monitor, wherein the appropriate calibration coefficient or calibration curve enables the monitor processor to calculate a physiological parameter.

11. The system of claim 10, wherein the sensor body comprises a current source configured to generate the current passed through the emitter.

12. The system of claim 10, wherein the processor is configured to periodically determine the voltage difference and to select the appropriate calibration coefficient or calibration curve for the wireless photolethysmography sensor during a monitoring session of a patient.

13. The system of claim 10, wherein the sensor processor is configured to determine a wavelength of light emitted by the emitter based on the voltage difference, and to select the appropriate calibration coefficient or calibration curve based on the wavelength.

14. The system of claim 10, wherein the monitor comprises a display, and the monitor processor is configured to instruct the display to display the physiological parameter.

15. The system of claim 10, wherein the sensor body comprises a detector configured to receive the light emitted by the emitter and to generate a signal based on the light received from the emitter, wherein the wireless transmitter is configured to wirelessly transmit the signal to the monitor, and the monitor processor is configured to calculate the physiological parameter using the signal and the appropriate calibration coefficient or calibration curve.

16. A method of operating a wireless photoplethysmography system, comprising:
passing a first current through an emitter positioned within a sensor body of a wireless photoplethysmography sensor to cause the emitter to emit light;
measuring, using a calibration circuit positioned within the sensor body, a first voltage present at an electrical input of the emitter and a second voltage present at the electrical output of the emitter;
determining, using a processor positioned within the sensor body, a voltage difference between the first voltage and the second voltage when the first current is passed through emitter;
selecting, using the processor, an appropriate calibration coefficient or calibration curve for the wireless photoplethysmography sensor from a plurality of calibration coefficients or calibration curves stored on a memory positioned within the sensor body based on the voltage difference, wherein each of the plurality of calibration coefficients or calibration curves corresponds to respective wavelengths of light that may be emitted by the emitter; and
transmitting, using a wireless transceiver, the appropriate calibration coefficient or calibration curve to a monitor to enable the monitor to calculate a physiological parameter.

17. The method of claim 16, comprising periodically repeating the steps of passing the first current through the emitter, measuring the first and second voltages, determining the voltage difference, selecting the appropriate calibration coefficient or calibration curve, and transmitting the appropriate calibration coefficient or calibration curve to the monitor during a monitoring session of a patient to periodically update the appropriate calibration coefficient or calibration curve to enable the monitor the calculate the physiological parameter.

18. The method of claim 16, comprising determining, using the processor, a wavelength of light emitted by the emitter based on the voltage difference, and selecting, using the processor, the appropriate calibration coefficient or calibration curve based on the wavelength.

19. The method of claim 16, comprising, using the processor, selecting the appropriate calibration coefficient or the calibration curve by directly correlating a value of the voltage difference to the plurality of calibration coefficients or calibration curves stored on the memory and without calculating a wavelength of light emitted by the emitter.

20. The method of claim 16, comprising:
passing a second current through the emitter to cause the emitter to emit light;
detecting, using a detector positioned within the sensor body, light emitted by the emitter in response to the second current passed through the emitter;
generating a signal, using the detector, based on the detected light; and
transmitting, using the wireless transceiver, the signal to the monitor to enable the monitor to calculate the physiological parameter using the signal and the appropriate calibration coefficient or calibration curve.
